(19) United States
(12) Patent Application Publication
(10) Pub. No.: US 2007/0232639 A1
(43) Pub. Date: Oct. 4, 2007

(54) Camptothecin Derivatives Conjugated in Position 20 with Integrin Antagonists

(76) Inventors: Claudio Pisano, Aprilia (IT); Giuseppe Giannini, Pomezia (RM) (IT); Maria Ornella Tinti, Rome (IT); Loredana Vesci, Rome (IT); Domenico Alloatti, Rome (IT); Sergio Penco, Milan (IT); Alma Dal Pozzo, Rome (IT); Ni Minghong, Milan (IT); Sabrina Dallavalle, Vimercate (MI) (IT); Lucio Merlini, Milan (IT); Franco Zunino, Milan (IT)

Correspondence Address: NIXON & VANDERHYE, PC, 901 NORTH GLEBE ROAD, 11TH FLOOR, ARLINGTON, VA, 22203, US

(21) Appl. No.: 11/596,189

(22) PCT Filed: May 4, 2005
(86) PCT No.: PCT/IT05/00261

§ 371 Date: Nov. 13, 2006

(30) Foreign Application Priority Data
May 13, 2004 (IT) RM2004A000241

(51) Int. Cl. A61K 31/437; A61K 31/4375; C07D 405/14

(52) U.S. Cl. 514/283; 546/276.7

(57) ABSTRACT

Compounds or formula (I) are described, in which the R and R₁ groups are as defined here below and include the condensation of the camptothecin molecule in position 20 with a cyclopeptide containing the RGD sequence. Said compounds are endowed both with high affinity for integrin receptors αᵥβ₃ and αᵥβ₅ and with selective cytotoxic activity on human tumour cell lines at micromolar concentrations.

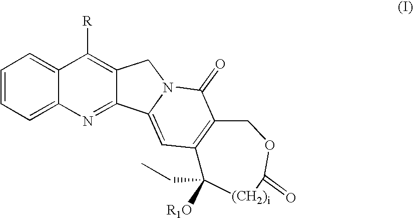

### FIELD OF THE INVENTION

The present invention relates to compounds with cytotoxic activity consisting of cyclopeptides containing the RGD sequence conjugated to camptothecin derivatives, methods for the preparation thereof, their use as medicaments and compositions containing them.

In particular, the compounds described in the present invention are endowed with both high affinity for integrins αᵥβ₃ and αᵥβ₅ and selective cytotoxic activity on human cell lines at micromolar concentrations.

### BACKGROUND OF THE INVENTION

Chemotherapeutic anticancer agents are the drugs with the most restrictive therapeutic window. In fact, since their cytotoxic activity is non-selective they may indiscriminately damage all the cells of the body with which they come into contact.

There currently exists the problem of directing the cytotoxic agent selectively against the tumour cells, allowing the agent to exert its activity without damaging the cells of the healthy surrounding tissues, or at least limiting the damage as much as possible.

It has been reported in the literature that blocking the integrins αᵥβ₃ and αᵥβ₅ by means of the use of selective cyclopeptides, the reference compound for which is regarded as cyclopentapeptide c(Arg-Gly-Asp-D-Phe-Val) (JACS 1997, 119, 1328-35; international patent application WO 97/06791), or by means of the use of monoclonal antibodies (Cell, 1994, 79, 1157-64) leads to the blocking of angiogenesis and to a reduction of tumour growth. In addition, antimetastatic effects have also been observed (J. Clin. Invest., 1995, 96, 1815). Brooks et al. (Science, 1994, 264, 569-71) reported that the endothelial cells of the tumour vasculature and the tumour cells themselves preferentially express integrin αᵥβ₃ compared to the quiescent cells of normal tissue. Among the compounds at an advanced stage of clinical development, we may mention c(Arg-Gly-Asp-D-Phe-MeVal), or EMD121974 or cilengitide.

Ruoslati and co-workers (Current Opinion in Oncology, 1998, 10, 560-5) showed that RGD analogues that bind to the tumour endothelium, once conjugated to the cytotoxic agent doxorubicin, form compounds that are more efficient and less toxic than doxorubicin alone. These authors also demonstrated, beyond any reasonable doubt, that the effect is attributable to the conjugation to RGD, inasmuch as the binding is antagonised by the free peptide itself (Arap, Pasqualini and Ruoslati, Science, 1998, 279, 377-380). Later, the same authors carried out other experiments consisting in chemically binding a pro-apoptotic peptide sequence to an RGD analogue, demonstrating that the new compounds were selectively toxic for angiogenic endothelial cells and had anticancer activity in mice (Ruoslati, Nature Medicine, 1999, 5, 1032-8).

Marcus et al., in international patent application WO 01/17563, describe specific anticancer activity for cytotoxic agents, such as camptothecin, conjugated by means of a spacer, consisting of one or more amino acids, to a non-peptidic inhibitor antagonist of integrins αᵥβ₃ and αᵥβ_{5.}

Aoki et al., Cancer Gene Therapy, 2001, 8, 783-787 describe the specific anticancer activity of a histidylated oligolysine conjugated to an RGD sequence, revealing a homing effect for tumours in mice.

The concept of binding at the cell surface mediated by integrins has been proposed for gene transport (Hart, et al., J. Biol. Chem., 1994, 269, 12468-12474).

It has now been found that the 7-iminomethyl or 7-oxymethyl camptothecin derivatives conjugated in position 20, possibly by means of suitable spacers, to cyclopeptide derivatives containing the RGD sequence are endowed with high, selective anticancer activity and can be advantageously used for the preparation of medicaments for the treatment of tumours.

By virtue of their selective cytotoxic activity on tumour cells, the compounds according to the present invention yield medicaments with fewer and less severe side effects.

### DESCRIPTION OF THE INVENTION

The subject of the present invention are camptothecin derivatives conjugated to cyclopeptide derivatives containing the RGD sequence. The resulting molecules conserve unaltered both the cytotoxic properties of the original camptothecins and integrin binding properties with affinity comparable to that observed with the non-conjugated cyclopeptides. The result of this combination is to favour the concentration of the cytotoxic agent in those cells that most express integrins of the αᵥβ₃ and αᵥβ₅ type (homing). The cytotoxic agent exerts its intracellular activity in the conjugated and/or free form through enzymatic or hydrolytic action.

The main object of the present invention are compounds of Formula I, as follows:

where:
i is 0 or 1; 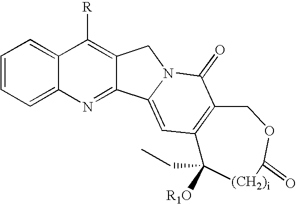
R is the —CH═N—(O)ₘ—R₂ group;
R₁ is the U—X—Y group;
where m is 0 or 1;
R₂ is selected from the group consisting of: saturated or unsaturated, linear or branched C₁-C₇ alkyl with the proviso that, when i is 0, R₂ is not t-Butyl; saturated or unsaturated C₃-C₁₀ cycloalkyl; C₆-C₁₄ aryl, aromatic or non-aromatic C₃-C₁₄ heterocyclic group, containing at least one heteroatom selected from the group consisting of O, N, S; saturated or unsaturated, linear or branched C₁-C₇ alkyl substituted with saturated or unsaturated C₃-C₁₄ cycloalkyl; C₆-C₁₄ aryl, aromatic or non-aromatic C₃-C₁₄ heterocyclic group, containing at least one heteroatom selected from the group consisting of O, N, S; a polyaminoalkyl of formula: —(CH₂)ₘ₁—NR₈—(CH₂)ₙ₁—NR₉—(CH₂—CH₂—CH₂—NR₉)ₚ₁—H, where m₁ and n₁, which may be the same or different, are an integer number from 2 to 6 and p₁ is an integer number from 0 to 3, and R₈ and R₉, which may be the same or different, are selected from the group consisting of H, linear or branched C₁-C₆ alkyl, Boc, Cbz, mono-saccharides such as 6-D-galactosyl or 6-D-glucosyl; each of the above-mentioned groups may possibly be substituted by one or more groups selected from the group consisting of —CN, —NO₂, —NH₂, —OH, —SH, —COOH, —COO-(alkyl)(C₁-C₅), —CONH-(alkyl)(C₁-C₅), —SO₃H; —SO₃-(alkyl)(C₁-C₅), where the alkyl group is linear or branched; a halogen atom;
U is either absent or one of the following groups —COCHR₁₀NH— or CON[(CH₂)ₙ₂NHR₇]—CH₂—, where R₁₀ is H or is selected from the group consisting of: linear or branched C₁-C₄ alkyl, optionally substituted with C₆-C₁₄ aryl or an amino-alkyl C₁-C₄; R₇ is H or linear or branched C₁-C₄ alkyl; n₂ is an integer number from 2 to 6;
X is absent or is H or is a group selected among the following: —CO CHR₃NH—, —CO CHR₆(CH₂)ₙ₃R₄—, —R₄—CH₂(OCH₂CH₂)ₙ₄OCH₂R₄—,
—R₄(Q)R₄—, —R₅ [Arg-NH(CH₂)ₙ₅CO]ₙ₆R₅—, —R₅—[N-guanidinopropyl-Gly]ₙ₆R₅—, in which n₃ is an integer number from 0 to 5, n₄ is an integer number from 0 to 50, n₅ is an integer number from 2 to 6, n₆ is an integer number from 2 to 7;
R₃ is H or linear or branched C₁-C₄ alkyl, optionally substituted with —COOH, —CONH₂, —NH₂ or —OH;
R₄ is selected from the group consisting of: —NH—, —CO—, —CONH—, —NHCO—;
R₅ is either absent or is the group —R₄(Q)R₄—;
R₆ is H or NH₂;
Q is selected from the group consisting of: linear or branched C₁-C₆ alkylene; linear or branched C₃-C₁₀ cycloalkylene; linear or branched C₂-C₆ alkenylene; linear or branched C₃-C₁₀ cyclo-alkenylene; C₆-C₁₄ arylene; arylene (C₆-C₁₄)-alkylene; (C₁-C₆), alkylene (C₁-C₆)-arylene (C₆-C₁₄); aromatic or non-aromatic heterocyclyl (C₃-C₁₄), containing at least one heteroatom selected from the group consisting of O, N, S;
Y is absent or H or is the following group c(Arg-Gly-Asp-AA₁-AA₂),
in which:
c means cyclic;
AA₁ is selected from the group consisting of: (D)-Phe, (D)-Trp, (D)-Tyr, (D)-2-naphthylAla, (D)-4-terbutyl-Phe, (D)-4,4′-biphenyl-Ala, (D)-4-CF₃-Phe, (D)-4-acetylamino-Phe;
AA₂ is selected from the group consisting of: NW—CH[(CH₂)ₙ₇—CO]—CO, NW—CH[(CH₂)ₙ₇—NH]—CO, NW—[4—(CH₂)ₙ₇—CO]-Phe, NW—[4—(CH₂)ₙ₇—NH]-Phe, [NW]-Gly, NW-Val, in which W is selected from H, linear or branched C₁-C₆ alkyl, —(CH₂)ₙ₇—COOH where n₇ is an integer number from 0 to 5, 4-carboxybenzyl, 4-aminomethylbenzyl;
with the proviso that X and Y cannot be both absent; the N₁-oxides, racemic mixtures, their single enantiomers, their single diastereoisomers, the forms E and Z, mixtures thereof, the pharmaceutically acceptable salts.

The compounds of Formula (I) may be prepared by the process described here below and exemplified for the preferred compounds according to the invention. This process constitutes a further subject of the invention.

Fundamentally, the compounds of Formula (I) which are the object of the present invention are prepared by means of the condensation of 7-substituted camptothecins or analogues thereof (indicated as “7-R—CP”), possibly functionalised via a suitable bridge (indicated as “U₁−X₁”), with a cyclopeptide derivative (indicated as “Y₁”).

The condensation reactions can be carried out according to one of the following reaction schemes:
7-R—CP+U₁−X₁−Y₁ or
7-R—CP+U₁+X₁+Y₁ or
7-R—CP−U₁+X₁−Y₁ or
7-R—CP−U₁+X₁+Y₁ or
7-R—CP−U₁−X₁+Y₁;
   where 7-R—CP represents a 7-substituted camptothecin or an analogue, in which R has the same meaning as defined in Formula (I), U₁, X₁ and Y₁ represent respectively the groups U, X and Y as defined in Formula I, possibly appropriately functionalised and protected so that the conjugated compounds of Formula I are obtained.

It must be pointed out that compounds of Formula (I) in which U, X or Y are absent are also active as antiproliferative or citotoxic agents and are therefore to be considered as part of the present invention.

These reactions are conducted using conventional methods, such as, for instance, those described in Journal of Controlled Release 2003, 91, 61-73; S. S. Dharap et al.; Journal of Medicinal Chem. 2003, 46, 190-3, R. Bhatt;

The camptothecin with a modified lactone ring (i=1) can be prepared in according to the procedure described in patent WO2003/101995, filed in the name of the present applicant.

The cyclopeptides Y₁ can be prepared according to conventional peptide synthesis techniques, as described in examples 4 to 6. The peptide synthesis can be accomplished either in the solid phase or in solution.

Once the desired cyclopeptide has been obtained, it will be used in the condensation reaction in its protected form, and the protective groups will be removed only after obtaining the final compound. The deprotection is done using known methods, e.g. acid conditions by means of the use of pure trifluoroacetic acid or in the presence of chlorinated organic solvents.

The camptothecin derivatives are obtained using methods which are common knowledge to experts of the field.

The compounds described in the present invention are topoisomerase I inhibitors and are therefore useful as medicaments, particularly for the treatment of diseases that benefit from the inhibition of said topoisomerase. In particular, the compounds according to the present invention exhibit antiproliferative activity, and therefore are used for their therapeutic properties, and possess physicochemical properties which make them suitable for formulation in pharmaceutical compositions.

The pharmaceutical compositions contain at least one compound of Formula (I) as an active ingredient, in an amount such as to produce a significant therapeutic effect. The compositions covered by the present invention are entirely conventional and are obtained using methods that are common practice in the pharmaceutical industry. According to the administration route opted for, the compositions will be in solid or liquid form and suitable for oral, parenteral or intravenous administration. The compositions according to the present invention contain, along with the active ingredient, at least one pharmaceutically acceptable-vehicle or excipient. Formulation adjuvants, such as, for example, solubilising agents, dispersing agents, suspension agents or emulsifying agents may be particularly useful.

The compounds of Formula (I) can also be used in combination with other active ingredients, such as, for example, anticancer agents or other drugs with antiparasitic or antiviral activity, both in separate forms and in a single dosage form.

The compounds according to the present invention are useful as medicaments with anticancer activity, e.g. in non-microcytoma and small-cell lung cancer, or in colorectal or prostate cancer, glioblastoma and neuroblastoma, cervical cancer, ovarian cancer, gastrointestinal carcinoma, carcinoma of the liver, Kaposi's sarcoma, renal carcinoma, sarcoma and osteosarcoma, testicular carcinoma, carcinoma of the breast, carcinoma of the pancreas, melanoma, carcinoma of the urinary bladder and of the head and neck. One of the advantages afforded by the compounds according to the present invention is the combination of antitopoisomerase activity, proper to the camptothecin portion of the molecule, and the integrin inhibiting activity, provided by the cyclopeptide portion of the molecule. The result is the possible combined action of the compounds according to the present invention which will be favourably received in the oncological sector by the experts operating in that sector. In fact, the cyclopeptide portion, containing the Arg-Gly-Asp sequence, not only directs the molecule against tumours expressing integrins, but, once the target has been reached, is capable of exerting multiple functions, ranging from the internalisation of the cytotoxic portion of the molecule to integrin inhibiting activity, with the resulting advantages, particularly in terms of the inhibition of tumour angiogenesis. The cyclopeptide portion, once separated from the camptothecin portion, is also capable of exerting its action at a distance from the site of the tumour, and therefore the compounds according to the present invention also prove useful in the prevention or treatment of metastatic forms.

The medicaments which are the subject of the present invention can also be used in the treatment of parasite diseases.

The following examples further illustrate the invention.

The abbreviations used are the following:
Aad (aminoadipic acid);
Amb (aminomethylbenzyl);
Amp (aminomethylphenylalanine);
Boc (ter-butoxycarbonyl);
CSA (camphosulfonic acid);
CTH (catalytic transfer hydrogenation);
DCC (dicyclohexylcarbodiimide);
DCM (dichloromethane);
DIEA (diisopropylethylamine);
DMF (dimethylformamide);
Dy(OTf)₃ dysprosium triflate;
Fmoc (9-fluorenylmethyloxycarbonyl);
HOBT (hydroxybenzotriazole);
NMP (N-methyl-pyrrolidone);
Pht phthaloyl);
Pmc (pentamethylchroman-6-sulphonyl);
TBTU (tetrafluoroborate-O-benzotriazol-1-yl-tetramethyluronium);
TFA (trifluoroacetic acid).

### EXAMPLES

### Example 1

### Synthesis of c(Arg(Pmc)-Gly-Asp(OtBu)-D-Phe-Amp) (protected ST2581)

1.587 mmol of Fmoc-Gly-Res (Res=Sasrin Resin®, Bachem) were suspended under stirring in 75 ml of DMF for 30 minutes, after which 18 ml of piperidine were added, continuing the stirring for a further 30 minutes. The resin, filtered and washed with DMF, was suspended in 50 ml of NMP (N-methyl-pyrrolidone) for 15 minutes, after which Fmoc-Arg(Pmc)-OH, HOBT, TBTU and DIEA were added (3.174 mmol of each); after 2 hours of stirring, the suspension was filtered and washed with DMF. After deprotection with piperidine, the coupling was repeated with the other amino acids in succession, operating each time as described above, namely: Fmoc-Amp(Cbz)-OH, Fmoc-D-Phe-OH, and Fmoc-Asp(OtBu)-OH. After the last deprotection of the Fmoc-N-terminal, the linear pentapeptide was released from the resin with 45 ml of 1% TFA in DCM. This was dissolved in approximately 1 l of CH₃CN, and 4.761 mmol of HOBT and TBTU and 10 ml of DIEA were added; the solution was kept under stirring for 30 minutes, the solvent was evaporated to a small volume and the precipitation of the product was completed with water. The filtered crude product was dissolved in 27 ml of a mixture of MeOH and DMF 1:1; 5 mmol of ammonium formiate and 0.55 g of 10% Pd/C were added and left under stirring at room temperature for 30 minutes. The suspension was filtered on celite and brought to dryness. The residue was purified by preparatory RP-HPLC (column: Alltima® C-18, Alltech; mobile phase 50% CH₃CN in water +0.1% TFA; retention time (Rt)=9.13 minutes). 483 mg of a white powder were obtained.

¹H-NMR (DMSO-d₆) δ 8.3, 8.07, 8.04, 7.90, 7.80, 7.33, 7.15, 7.07, 4.62, 4.50, 4.35, 4.12, 4.01, 3.15, 3.03, 2.96-2.65, 2.58, 2.48, 2.32, 2.02, 1.75, 1.50, 1.35, 1.23.

Molecular mass (Maldi-Tof): 973

### Example 2

### Synthesis of c(Arg(Pmc)-Gly-As-p(OtBu)-D-Phe-Aad) (protected ST2650)

0.69 mmol of Fmoc-Gly-Res were treated exactly as described in example 1, with the difference that in this case the third and fourth amino acids were added in the form of dipeptide Fmoc-D-Phe-Aad(OBzl)-OH. After deprotection by means of CTH, and purification of the crude product with preparatory RP-HPLC (mobile phase: 66% CH₃CN in water +0.1% TFA; Rt=17.29 minutes), 187 mg of pure peptide were obtained.

¹H-NMR (DMSO-d₆) δ 7.23, 4.58, 4.20-3.90, 3.28, 3.05, 2.99, 2.85, 2.74-2.35, 2.15, 2.05, 1.85-1.25.

Molecular mass (Maldi-Tof): 940

### Example 3

### Synthesis of c(Arg(Pmc)-Gly-Asp(OtBu)-D-Phe-N-Me-Amp) (protected ST2700)

To a suspension of Fmoc-Phe(4-Pht-N—CH₂)—COOH in anhydrous toluene brought to reflux 2 eq of CSA and 20 eq of paraformaldehyde were added, divided into 4 portions at intervals of 15 minutes. The mixture was left to cool, diluted with 120 ml of toluene and washed with 5% NaHCO₃ and water. After evaporation of the solvent, the residue was dissolved in 15 ml of CHCl₃+15 ml of TFA+700 μl of Et₃SiH; the mixture was left in the dark to stir for 42 hours. After evaporation of the solvent, the residue was purified by filtration on silica gel. Overall yield: 90%.

The linear peptide was synthesized in solid phase as described in example 1, inserting Fmoc-N-Me-Phe-(4-Pht-N—CH₂)—COOH as the third amino acid, prepared as described above. In this case the deprotections of N-Fmoc-terminal on resin were carried out with 30% diisopropylamine (300 eq) in solution in DMF (owing to the presence of phthalimide). After cyclisation, 500 mg of the peptide were dissolved hot in 10 ml of absolute EtOH, to which 0.9 ml of a solution of NH₂—NH₂.H₂O 1 M in ethanol was added. After heating at reflux for 2 hours, the solvent was evaporated and the residue taken up with 10 ml of DCM+10 ml of Na₂CO₃ solution under vigorous shaking. The crude final product was recovered from the organic phase after evaporation and purified by preparatory RP-HPLC (mobile phase: 52% CH₃CN in water+0.1% TFA; Rt=10 minutes).

¹H-NMR (CDCl₃) δ 8.29-7.66, 7.38-7.07, 4.95-4.77, 4.09, 3.41, 3.05-2.81, 2.51, 2.05, 1.74, 1.40, 1.26.

Molecular mass (Maldi-Tof): 987

### Example 4

### Synthesis of c[Arg(Pmc)-Gly-As-p(OtBu)-D-Phe-Am-p(CO—(CH₂)₂ —COOH)] (protected ST2649)

120 mg of cyclopeptide c[Arg(Pmc)-Gly-Asp(OtBu)-D-Phe-Amp].TFA (prepared as described in example 1) were dissolved in 3.6 ml of a mixture of DCM-DMF 2:1, together with a stoichiometric amount of TEA and succinic anhydride. After 1 hour the reaction mixture was diluted with 30 ml of DCM and washed with water. The organic phase, dried and concentrated, yielded a residue of 100 mg of pure product.

Analytical RP-HPLC: column: Purosphere STAR®, Merck; mobile phase: 45% CH₃CN in water+0.1% TFA; Rt=13.17 minutes.

¹H-NMR(DMSO-d₆) δ 8.20-7.75, 7.19-7.02, 4.58, 4.45, 4.36, 4.30, 4.20, 4.05, 3.00, 2.97-2.57, 1.83, 1.62, 1.32.

Molecular mass (Maldi-Tof): 1073

### Example 5

### Synthesis of c(Arg(Pmc)-Gly-Asp(OtBu)-D-Phe-N-Amb-Gly) (protected ST2701)

To a solution of 1-22 mmol of Boc-monoprotected p-xylylenediamine in 6 ml of THF were added 1.83 mmol of TEA and, dropwise, a solution of 1.22 mmol of benzyl bromoacetate in 2 ml of THF. The mixture was left under stirring overnight, after which the solvent was evaporated and the residue purified on a flash column (CHCl₃-EtOAc, 9:1). 0.69 mmol of N-(4-Boc-NH-CH₂-benzyl)-glycine benzylester were obtained.

250 mg of Fmoc-D-Phe-OH were dissolved in 27 ml of DCM and 40 μl of diphosgene and 230 μl of sym-collidine were added; after 15 minutes 190 mg of the previously prepared ester were added, dissolved in 3 ml of DCM. After 3 hours, 80 μl of N-Me-piperazine were added to the reaction mixture and stirred for 10 minutes, after which the mixture was diluted with 10 ml of DCM and extraction was done with water, HCl 0.5N, water, 5% NaHCO₃ and water. After evaporation of the solvent, the residue was purified by flash chromatography on silica gel (DCM-EtOAc, 9:1). Yield: 80%.

To 100 mg of the product thus obtained, dissolved in 6 ml of MeOH, were added 76 μl of AcOH and 42 mg of HCOONH₄, and the mixture cooled to 0° C., and 50 mg of 10% Pd/C were added. After 30 minutes, the reaction mixture was filtered on celite. The filtrate was brought to dryness and purified on a flash column (CHCl₃—MeOH 9:1). Yield: 90%.

190 mg of the product thus obtained were dissolved in 1.2 ml of TFA and brought to dryness (deprotection of Boc); the residue was redissolved in 9 ml of 10% Na₂CO₃+6 ml of dioxane, cooled to 0° C. and a solution of 120 μl of benzyloxycarbonyl chloride diluted with 3 ml of dioxane was added dropwise. After 1 hour stirring at room temperature, evaporation was carried out under vacuum to a small volume, after which the mixture was diluted with water, the pH was reduced to 1 with HCl and extraction was done with EtOAc. After evaporation of the solvent, the residue was purified by filtration on silica gel, washing with CHCl₃—MeOH (8:2). Pure dipeptide yield: 82%.

0.69 mmol of Fmoc-Gly-Res were treated as described in example 1. After Arg, the previously prepared dipeptide Fmoc-D-Phe-N(4-Cbz-NH-CH₂-benzyl)-Gl was added in sequence. After deprotection of Cbz by means of CTH, the crude product c(Arg(Pmc)-Gly-Asp(OtBu)-D-Phe-N-Amp-Gly) was purified by preparatory RP-HPLC (mobile phase: 50% CH₃CN in water+0.1% TFA; Rt=10.5 minutes).

¹H-NMR (DMSO-d₆) δ 8.29-7.66, 7.44-6.90, 5.15, 4.72-4.18, 4.20, 4.05-3.32, 3.15, 3.06, 2.70, 2.51, 2.49, 2.01, 1.80-1.35, 1.49, 1.35, 1.23.

Molecular mass (Maldi-Tof): 973

### Example 6

### Synthesis of c(Arg(Pmc)-Gly-Asp)(OtBu)-D-Phe-Amo(CO—CH₂—(OCH₂CH₂)ₙ—O—CH₂—COOH))

To a solution of 200 mg of c(Arg(Pmc)-Gly-Asp(OtBu)-D-Phe-Amp).TFA (obtained as described in example 1) in 4 ml of a 3:1 DCM-DMF mixture was added a substantial excess of glycol diacid. DIEA (3 eq) and DCC (2 eq) were added to the same solution. The mixture was left under stirring overnight, after which it was diluted with DCM and washed with water.

The crude product was recovered by evaporating the organic phase and purified by flash chromatography (mobile phase: CHCl₃—MeOH 7:3+1% AcOH); the fractions containing the product were pooled, washed with water, dehydrated and brought to dryness, and yielded a residue of 157 mg of pure product.

Analytical RP-HPLC: (column: Purosphere STAR®, Merck; mobile phase: 50% CH₃CN 50% in water+0.1% TFA; Rt=10.96)

¹H-NMR (DMSO-d₆) δ 8.35-7.92, 7.20-7.00, 4.65, 4.50, 3.94, 3.60-3.45, 3.00-2.60, 2.55, 2.45, 2.30, 2.00, 1.70, 1.50, 1.30, 1.20.

Molecular mass (Maldi-Tof): corresponding to the different glycols used of various molecular weights.

### Synthesis of Camptothecin Derivatives—Compounds of Formula (I)

### Example 7

### Synthesis of 7-R—CP (20-O-Val)

One mmol of 7-R—CP, 0.6 mmol of Dy(OTf)₃, 3 mmol of dimethylaminopyridine and 3 mmol of Boc-Val-OH were suspended in 15 ml dianhydrous CH₂Cl₂ and brought to −10° C.; after 30 minutes 3.1 mmol of DCC were added and after another 30 minutes at −10° C. the reaction mixture was brought to room temperature. After 2 hours the reaction was diluted with another 20 ml of CH₂Cl₂, washed with 1N HCl, with Na—HCO₃ and dried on Na₂SO₄. The crude product was purified by chromatography on SiO₂ with CH₂Cl₂/MeOH 97:3.

The intermediate product [N-Boc] was deprotected in DCM/TFA (75/25) at 0° C., with a quantitative yield. The compound thus obtained was used to bind the cyclopeptides Y₁ directly or as a further intermediate, which can be used to bind a second residue (see examples 8-9).

### Example 7 bis

### Synthesis of 7-[CH═N—O—CH₂CH₂—(N)-Morpholine]-CP (20-O-Val)—(ST2896)

Example 7 was carried out exactly as described above to obtain the compound of Formula (I), in which R is the group —CH═N—OCH₂CH₂-Morpholine (i.e. in which m is 0 and R₂ is ethylene(N)morpholine), i is 0 and R₁ is Val. The intermediate [N-Boc]-protected product showed the following experimental data:

¹H-NMR (CDCl₃) δ 9.09, 8.20-8.25, 7.81-7.88, 7.68-7.75, 7.32, 5.65-5.74, 5.38-5.47, 5.41, 5.00-5.04, 4.57-4.59, 4.31-4.38, 3.77-3.81, 2.89-2.94, 2.66-2.71, 2.14-2.38, 1.54, 0.92-1.07.

Molecular mass (ESI⁻): 702.5 (M−1)

This intermediate was deprotected in DCM/TFA (75/25) at 0° C., to give ST2896 with a quantitative yield.

¹H-NMR (CDCl₃) δ 9.09, 8.20-8.25, 7.81-7.88, 7.68-7.75, 7.32, 5.65-5.74, 5.38-5.47, 5.41, 5.00-5.04, 4.57-4.59, 4.31-4.38, 3.77-3.81, 2.89-2.94, 2.66-2.71, 2.14-2.38, 0.92-1.07.

Molecular mass (ESI⁺): 604.7 (M+1)

### Example 8

### Synthesis of 7-R—CP (20-O-Val-Asp)

One mmol of the compound obtained in example 7 and 3.7 mmol of DIPEA were added in that order to a solution of 1.2 mmol of suitably protected aspartic acid, 1.8 mmol of HOBt and 1.4 mmol of EDC in DMF at 0° C. The reaction mixture was left overnight at room temperature before being partitioned between water and dichloromethane, and the crude product thus obtained was purified by chromatography on SiO₂ with CH₂Cl₂/MeOH 96:4 to give the product as a yellow solid with a yield of 66%.

### Deprotection of the Carboxyl Group

The benzylester was hydrogenolysed with H₂/10% Pd-C at 20 psi with a yield of 70% after purification with CH₂Cl₂/MeOH 94:6.

### Example 8 bis

### Synthesis of 7-[CH═N—O—CH₂CH₂-Morpholine]-CP (20-O-Val-Asp)—(ST3240)

Example 8 was carried out exactly as described above to obtain the compound of Formula (I), in which R is the group —CH═N—OCH₂CH₂-Morpholine (i.e. in which m is 0 and R₂ is ethylene(N)morpholine), i is 0 and R₁ is Val-Asp. The intermediate [N-Boc]-protected product showed the following experimental data:

¹H-NMR (CDCl₃) δ 9.09, 8.18-8.30, 7.66-7.83, 7.24-7.37, 7.19, 5.72-5.91, 5.63-5.72, 5.38-5.46, 5.40, 4.70-4.72, 4.49-4.59, 3.75-3.80, 3.10-3.21, 2.82-2.87, 2.58-2.66, 2.11-2.39, 1.74-1.88, 0.92-1.06.

Molecular mass (ESI⁻): 641.6 (M−1)

This intermediate was deprotected in DCM/TFA (75/25) at 0° C., to give ST3240 with a quantitative yield.

¹H-NMR (CDCl₃) δ 9.09, 8.18-8.30, 7.66-7.83, 7.24-7.37, 7.19, 5.72-5.91, 5.63-5.72, 5.38-5.46, 5.40, 4.70-4.72,4.49-4.59, 3.75-3.80, 3.10-3.21, 2.82-2.87, 2.58-2.66, 2.11-2.39, 1.74-1.88, 0.92-1.06.

Molecular mass (ESI⁺): 543.7 (M+1)

### Example 9

### Synthesis of 7-R—CP (20—O-Val-COCH₂CH₂COOH)

One mmol of the deprotected compound obtained in example 7 was dissolved in 10 mL of anhydrous pyridine and after bringing the solution to 0° C., 2.5 mmol of succinic anhydride were added: the mixture was restored to room temperature for 1 hour. The solvent was removed, the residue was taken up with CH₂Cl₂ and the organic phase was washed with 0.5N HCl. The crude product was purified by chromatography on SiO₂ with CH₂Cl₂/MeOH 95:5 to give the expected product as a yellow solid with a yield of 90%.

### Synthesis of Camptothecin Derivatives Conjugated to Cyclopeptides—Compounds of Formula (I)

### Example 10

### Synthesis of 20—O—(7-R—CP)-c(Arg(Pmc)-Gly-As-p(OtBu)-D-Phe-Amp) coniugates

To a solution of 1.2 mmol of the compound obtained in examples 8 or 9 in anhydrous DMF cooled to 0° C. were added 2.1 mmol of HOBt and 1.4 mmol of EDC and the resulting mixture was stirred for 30 minutes before adding 1 mmol of protected ST2581 prepared in Example 1 and DIPEA in sequence. After being left overnight the mixture was partitioned between water and dichloromethane and the organic phase was then dried on Na₂SO₄ and the crude product purified by chromatography on SiO₂ with CH₂Cl₂/MeOH 92:8 to give the expected products, with a yield ranging from 55% to 65%.

### Deprotection of Conjugated Products

The final deprotection was done with CH₂Cl₂/TFA 1:1 for 2 hours bringing the mixture from 0° C. to room temperature; this operation was followed by a step ion-exchange resin which gave the products as hydrochlorides.

### Example 10 bis

### Synthesis of 20—O—[7—(CH=N—O—CH₂CH₂-Morpholine)]-CP)-c(Arg(Pmc)-Gly-Asp(OtBu)-D-Phe-Amp) conjugate—(ST3241)

Example 10 was carried out exactly as described above and the conjugation between protected ST2581 and the camptothecin derivative ST3240 prepared in Example 8bis gave the compound named ST3241, having the following properties:

Molecular mass (ESI⁺): 1367.5 (M+1)

### Example 11

### Biological Results

### Binding to Integrin αᵥβ₃ Receptors

The purified αᵥβ₃ receptor (Chemicon, cat. CC1020) was diluted in buffer (20 mM Tris, pH 7.4, 150 mM NaCl, 2 mM CaCl₂, 1 mM MgCl₂, 1 mM MnCl₂) at a concentration of 0.5 μg/ml. An aliquot of 100 μl was added to 96-well plates and incubated overnight at +4° C. Plates were washed once with buffer (50 mM Tris, pH 7, 4, 100 mM NaCl, 2 mM CaCl₂, 1 mM MgCl₂, 1 mM MnCl₂, 1% bovine serum albumin) and then incubated for another 2 hours at room temperature. Plates were washed twice with the same buffer and incubated for 3 hours at room temperature with the radioactive ligand [¹²⁵I]echistatin (Amersham Pharmacia Biotech) 0.05 nM in the presence of competition ligands. At the end of incubation, the wells were washed and the radioactivity determined using a gamma counter (Packard). Non-specific binding of the ligand was determined in the presence of excess cold echistatin (1 μM).

### Binding to Integrin αᵥβ₅ Receptors

The purified αᵥβ₅ receptor (Chemicon, cat. CC1020) was diluted in buffer (20 mM Tris, pH 7.4, 150 mM NaCl, 2 mM CaCl₂, 1 mM MgCl₂, 1 mM MnCl₂) at a concentration of 1 μg/ml. An aliquot of 100 μl was added to 96-well plates and incubated overnight at +4° C. Plates were washed once with buffer (50 mM Tris, pH 7.4, 100 mM NaCl, 2 mM CaCl₂, 1 mM MgCl₂, 1 mM MnCl₂, 1% bovine serum albumin) and then incubated for another 2 hours at room temperature. Plates were washed twice with the same buffer and incubated for 3 hours at room temperature with the radioactive ligand [¹²⁵I]echistatin (Amersham Pharmacia Biotech) 0.15 nM in the presence of competition ligands. At the end of incubation, the wells were washed and the radioactivity determined using a gamma counter (Packard). Non-specific ligand binding was determined in the presence of excess cold echistatin (1 μM).

### Evaluation of IC₅₀ Parameters

The affinity of the products for vitronectin receptors was expressed as IC₅₀ value±SD, i.e. as the concentration capable of inhibiting 50% of the specific radioligand-receptor binding. The IC₅₀ parameter was elaborated using “ALLFIT” software.

### Results

All the RGD peptides examined showed significant affinity for αᵥβ₃ and αᵥβ₅ integrin receptors with an IC₅₀ value of the order of nanomoles. In particular, the most active in inhibiting echistatin binding to the αᵥβ₃ integrins was ST2581 (IC₅₀=1.7 nM).

TABLE 1
Affinity of RGD peptides for vitronectin αᵥβ₃ and αᵥβ₅ receptors

| | |
|---|---|
| αᵥβ₃ | αᵥβ₅ |

| | |
|---|---|
| Compound | IC₅₀ ± SD (nM) |

| | | |
|---|---|---|
| ST2581 | 1.7 ± 0.1 | 3.4 ± 0.1 |

1. Compounds of Formula I, as follows: 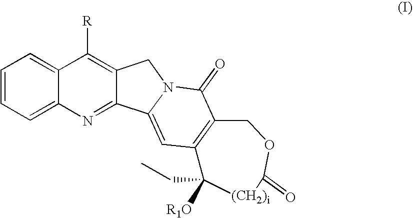
where:
i is 0 or 1;
R is the —CH═N—(O)ₘ—R₂ group;
R₁ is the U—X—Y group;
where m is 0 or 1;
R₂ is selected from the group consisting of: saturated or unsaturated, linear or branched C₁-C₇ alkyl with the proviso that, when i is 0, R₂ is not t-Butyl; saturated or unsaturated C₃-C₁₀ cycloalkyl; C₆-C₁₄ aryl, aromatic or non-aromatic C₃-C₁₄ heterocyclic group, containing at least one heteroatom selected from the group consisting of O, N, S; saturated or unsaturated, linear or branched C₁-C₇ alkyl substituted with saturated or unsaturated C₃-C₁₀ cycloalkyl; C₆-C₁₄ aryl, aromatic or non-aromatic C₃-C₁₄ heterocyclic group, containing at least one heteroatom selected from the group consisting of O, N, S; a polyaminoalkyl of formula: —(CH₂)ₘ₁—NR₈—(CH₂)ₙ₁—NR₉—(CH₂—CH₂—CH₂—NR₉)ₚ₁—H, where m₁ and n₁, which may be the same or different, are an integer number from 2 to 6 and p₁ is an integer number from 0 to 3, and R₈ and R₉, which may be the same or different, are selected from the group consisting of H, linear or branched C₁-C₆ alkyl, Boc, Cbz, monosaccharides such as 6-D-galactosyl or 6-D-glucosyl; each of the above-mentioned groups may possibly be substituted by one or more groups selected from the group consisting of —CN, —NO₂, —NH₂, —OH, —SH, —COOH, —COO-(alkyl)(C₁-C₅), —CONH-(alkyl)(C₁-C₅), —SO₃H; —SO₃-(alkyl)(C₁-C₅), where the alkyl group is linear or branched; a halogen atom;
U is either absent or one of the following groups —COCHR₁₀NH— or CON[(CH₂)ₙ₂NHR₇]—CH₂—, where R₁₀ is H or is selected from the group consisting of: linear or branched C₁-C₄ alkyl, optionally substituted with C₆-C₁₄ aryl or an amino-alkyl C₁-C₄; R₇ is H or linear or branched C₁-C₄ alkyl; n₂ is an integer number from 2 to 6;
X is absent or is H or is a group selected among the following: —COCHR₃NH—, —COCHR₆(CH₂)ₙ₃R₄—, —R₄—CH₂(OCH₂CH₂)ₙ₄OCH₂R₄—,
—R₄(Q)R₄—, —R₅[Arg-NH(CH₂)ₙ₅CO]ₙ₆R₅—, —R₅—[N-guanidinopropyl-Gly]ₙ₆R₅—, in which n₃ is an integer number from 0 to 5, n₄ is an integer number from 0 to 50, n₅ is an integer number from 2 to 6, n₆ is an integer number from 2 to 7;
R₃ is H or linear or branched C₁-C₄ alkyl, optionally substituted with —COOH, —CONH₂, —NH₂ or —OH;
R₄ is selected from the group consisting of: —NH—, —CO—, —CONH—, —NHCO—;
R₅ is either absent or is the group —R₄(Q)R₄—;
R₆ is H or NH₂;
Q is selected from the group consisting of: linear or branched C₁-C₆ alkylene; linear or branched C₃-C₁₀ cycloalkylene; linear or branched C₂-C₆ alkenylene; linear or branched C₃-C₁₀ cyclo-alkenylene; C₆-C₁₄ arylene; arylene (C₆-C₁₄)-alkylene; (C₁-C₆), alkylene (C₁-C₆)-arylene (C₆-C₁₄); aromatic or non-aromatic heterocyclyl (C₃-C₁₄), containing at least one heteroatom selected from the group consisting of O, N, S;
Y is absent or H or is the following group c(Arg-Gly-Asp-AA₁-AA₂), in which:
c means cyclic;
AA₁ is selected from the group consisting of: (D)-Phe, (D)-Trp, (D)-Tyr, (D)-2-naphthylAla, (D)-4-terbutyl-Phe, (D)-4,4′-biphenyl-Ala, (D)-4-CF₃-Phe, (D)-4-acetylamine-Phe;
AA₂ is selected from the group consisting of: NW—CH[(CH₂)ₙ₇—CO]—CO, NW—CH[(CH₂)ₙ₇—NH]—CO, NW—[4-(CH₂)ₙ₇—CO]-Phe, NW—[4—(CH₂)ₙ₇—NH]-Phe, [NW]-Gly, NW-Val, in which W is selected from H, linear or branched C₁-C₆ alkyl, —(CH₂)ₙ₇—COOH where n₇ is an integer number from 0 to 5, 4-carboxybenzyl, 4-aminomethylbenzyl;
with the proviso that X and Y cannot be both absent;
the N₁-oxides, racemic mixtures, their single enantiomers, their single diastereoisomers, the forms E and Z, mixtures thereof, the pharmaceutically acceptable salts.

2. Compounds according to claim 1, in which m is 1.

3. Compounds according to claim 2, in which R₂ is saturated or unsaturated, linear or branched C₁-C₃ alkyl.

4. Process for the preparation of compounds according to claim 1, according to one of the following reaction schemes:
7-R—CP+U₁−X₁−Y₁ or
7-R—CP+U₁+X₁+Y₁ or
7-R—CP−U₁+X₁−Y₁ or
7-R—CP−U₁+X₁+Y₁ or
7-R—CP−U₁−X₁+Y₁;
where 7-R—CP represents a 7-substituted camptothecin or an analogue thereof, in which R has the same meaning as defined in Formula (I), U₁, X₁ and Y₁ represent respectively the groups U, X and Y as defined in Formula (I), eventually appropriately functionalised and protected.

5. Pharmaceutical composition containing at least one compound according to claim 1 as the active ingredient in a mixture with at least one pharmaceutically acceptable excipient and/or vehicle.

6. Use of the compounds according to claim 1 for the preparation of medicaments.

7. Use of the compounds according to claim 1 for the preparation of a medicament endowed with topoisomerase 1 inhibiting activity.

8. Use according to claim 7 for the preparation of a medicament with anticancer activity.

9. Use according to claim 8, in which said medicament is useful for the treatment of non-microcytoma and small-cell lung cancer, colorectal tumours, prostate cancer, glioblastoma and neuroblastoma, cervical cancer, ovarian carcinoma, gastrointestinal carcinoma, carcinoma of the liver, Kaposi's sarcoma, renal carcinoma, sarcoma and osteosarcoma, testicular carcinoma, carcinoma of the breast, carcinoma of the pancreas, melanoma, carcinoma of the urinary bladder and of the head and neck.

10. Use of the compounds according to claim 1 for the preparation of a medicament useful for the prevention or treatment of meta-static forms.

11. Use according to claim 7 for the preparation of a medicament with antiparasite activity.

12. Use according to claim 7 for the preparation of a medicament with antiviral activity.
